(19) United States
(12) Patent Application Publication
(10) Pub. No.: US 2007/0181135 A1
(43) Pub. Date: Aug. 9, 2007

(54) Adjustable headgear assembly for sleep apnea patients

(75) Inventor: Jason M. Baker, Charlotte, NC (US)

Correspondence Address: Schwatz Law Firm, P.C.;Suite 530, SouthPark Towers, 6100 Fairview Road, Charlotte, NC, 28210, US

(73) Assignee: Headwear, LLC

(21) Appl. No.: 11/350,237

(22) Filed: Feb. 8, 2006

(51) Int. Cl. A61F 5/56

(52) U.S. Cl. 128/848

(57) ABSTRACT

A headgear assembly is adapted for wear by a sleep apnea patient. The headgear assembly is designed for holding a lower jaw of the patient substantially closed during sleep. The headgear assembly includes a flexible crown and chin strap. The crown resides atop a head of the patient, and comprises a crown-encircling strap and a crown opening defined by the strap. The chin strap has first and second ends attached to the crown, and is adapted for engaging a chin of the patient to support the lower jaw.

### TECHNICAL FIELD AND BACKGROUND OF THE INVENTION

This invention relates to an adjustable headgear assembly applicable for use by a sleep apnea patient to help support the lower jaw and keep the patient's mouth substantially closed during sleep. Mouth breathing during sleep causes the jaw to drop and reduces the diameter of the pharyngeal airway, increasing the likelihood of obstructive sleep apnea. Obstructive sleep apnea occurs when air cannot flow into or out of the patient's nose or mouth, although efforts to breathe continue.

Sleep apnea is a serious, potentially life-threatening breathing disorder characterized by brief interruptions of breathing during sleep. In a given night, the number of involuntary breathing pauses or “apneic events” may be as high as 20 to 30 or more per hour. These breathing pauses are almost always accompanied by snoring between apnea episodes, although not everyone who snores has this condition. Sleep apnea can also be characterized by choking sensations. The frequent interruptions of deep, restorative sleep often lead to early morning headaches and excessive daytime sleepiness.

Certain mechanical and structural problems in the airway cause the interruptions in breathing during sleep. In some people, apnea occurs when the throat muscles and tongue relax during sleep and partially block the opening of the airway. When the muscles of the soft palate at the base of the tongue and the uvula relax and sag, the airway becomes blocked, making breathing labored and noisy and even stopping it altogether. Sleep apnea also can occur in obese people when an excess amount of tissue in the airway causes it to be narrowed. With a narrowed airway, the person continues his or her efforts to breathe, but air cannot easily flow into or out of the nose or mouth. Unknown to the person, this results in heavy snoring, periods of no breathing, and frequent arousals causing abrupt changes from deep sleep to light sleep.

The specific therapy for sleep apnea is tailored to the individual patient based on medical history, physical examination, and the results of polysomnography. Medications are generally not effective in the treatment of sleep apnea. Dental appliances that reposition the lower jaw and the tongue have been helpful to some patients with mild sleep apnea. Possible side effects include damage to teeth, soft tissues, and the jaw joint. Some patients with sleep apnea may need surgery. Although several surgical procedures are used to increase the size of the airway, none of them is completely successful or without risks. More than one procedure may need to be tried before the patient realizes any benefits.

The present invention is a non-medicinal and non-invasive technique for treating sleep apnea. Unlike other aids designed to keep the mouth closed while sleeping, the invention is readily applied to and removed from the patient, comfortable to wear, securely holds the lower jaw closed, and will resist inadvertent shifting throughout the night.

### SUMMARY OF INVENTION

Therefore, it is an object of the invention to provide a headgear assembly especially designed for supporting the lower jaw and keeping the mouth substantially closed during sleep.

It is another object of the invention to provide a headgear assembly which is readily adjustable to fit each wearer.

It is another object of the invention to provide a headgear assembly which resists inadvertent shifting during wear.

It is another object of the invention to provide a headgear assembly is comfortable to wear.

It is another object of the invention to provide a headgear assembly which may be used in combination with a CPAP machine to limit loss of air through the mouth during CPAP treatment.

It is another object of the invention to provide a headgear assembly which incorporates a head crown-encircling strap which comfortably distributes any pressure against the head during wear.

It is another object of the invention to provide a method for holding a lower jaw of a sleep apnea patient substantially closed during sleep.

These and other objects of the present invention are achieved in the preferred embodiments disclosed below by providing a headgear assembly adapted for wear by a sleep apnea patient. The headgear assembly is designed for holding a lower jaw of the patient substantially closed during sleep. The headgear assembly includes a flexible crown and chin strap. The crown resides atop a head of the patient, and comprises a crown-encircling strap and a crown opening defined by the strap. The chin strap has first and second ends attached to the crown, and is adapted for engaging a chin of the patient to support the lower jaw.

According to another preferred embodiment of the invention, means are provided for adjusting the chin strap relative to the crown to increase or decrease pressure against the chin of the patient.

According to another preferred embodiment of the invention, the crown has a fastener ring adapted for adjustably receiving the first end of the chin strap.

According to another preferred embodiment of the invention, the first end of the chin strap includes complementary hook and loop fasteners. The chin strap is adjustably attached to the crown by inserting the first end through the fastener ring, and then folding the first end over to releasably mate the hook and loop fasteners.

According to another preferred embodiment of the invention, the crown has a second fastener ring receiving the second end of the chin strap.

According to another preferred embodiment of the invention, the second end of the chin strap includes complementary hook and loop fasteners. The chin strap is adjustably attached to the crown by inserting the second end through the second fastener ring, and then folding the second end over to releasably mate the hook and loop fasteners.

According to another preferred embodiment of the invention, the chin strap is constructed of a launderable foam material.

Preferably, the chin strap further includes elastic yarns.

According to another preferred embodiment of the invention, the chin strap defines a chin opening adapted for receiving the chin of the patient.

According to another preferred embodiment of the invention, the crown is constructed of a launderable foam material.

Preferably, the crown further includes elastic yarns.

According to another embodiment, the invention is a method for holding a lower jaw of a sleep apnea patient substantially closed during sleep. The method includes the steps of arranging a flexible crown atop a head of the patient. The crown comprises a crown-encircling strap and a crown opening defined by the strap. A flexible chin strap attached to the crown is located adjacent a chin of the patient to support the lower jaw.

According to another preferred embodiment of the invention, the method further comprises adjusting the chin strap relative to the crown to increase or decrease pressure against the chin of the patient.

### BRIEF DESCRIPTION OF THE DRAWINGS

Some of the objects of the invention have been set forth above. Other objects and advantages of the invention will appear as the description proceeds when taken in conjunction with the following drawings, in which:

FIGS. 1 and 2 are perspective views of a headgear assembly according to one preferred embodiment of the present invention, and showing the assembly in place on the head of a patient;

FIG. 3 is a further perspective view of the headgear assembly;

FIG. 4 is a view of the crown strap laid flat, and detached from the chin strap; and

FIG. 5 is a view of the chin strap laid flat, and detached from the crown strap.

### DESCRIPTION OF THE PREFERRED EMBODIMENT AND BEST MODE

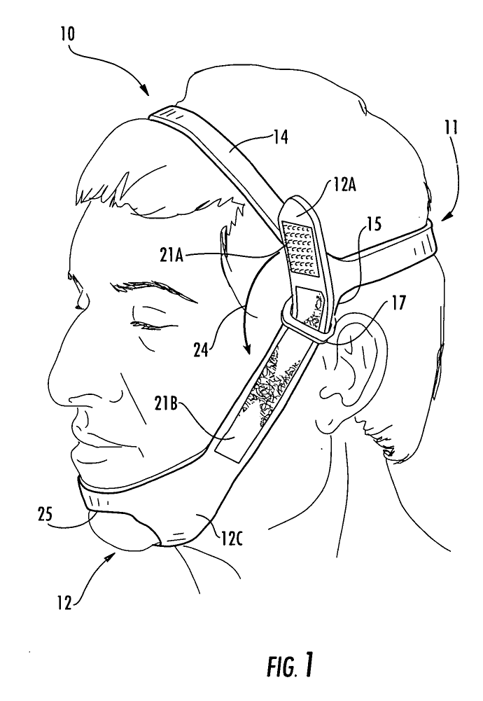

Referring now specifically to the drawings, a headgear assembly according to the present invention is illustrated in FIGS. 1 and 2, and shown generally at reference numeral 10. The headgear assembly 10 is especially applicable for wear by a sleep apnea patient to hold the lower jaw and mouth of the patient substantially closed during sleep, thereby urging the patient to breathe through the nose. Mouth breathing during sleep causes the jaw to drop and reduces the diameter of the pharyngeal airway, increasing the likelihood of obstructive sleep apnea. Obstructive sleep apnea occurs when air cannot flow into or out of the patient's nose or mouth, although efforts to breathe continue. The headgear assembly 10 may also be used in combination with a continuous positive airway pressure (CPAP) machine which provides a low flow of air pressure through the airway by way of a nasal mask. While undergoing CPAP therapy, the patient must keep the mouth closed in order to limit the escape of air and risk losing the effect of CPAP treatment.

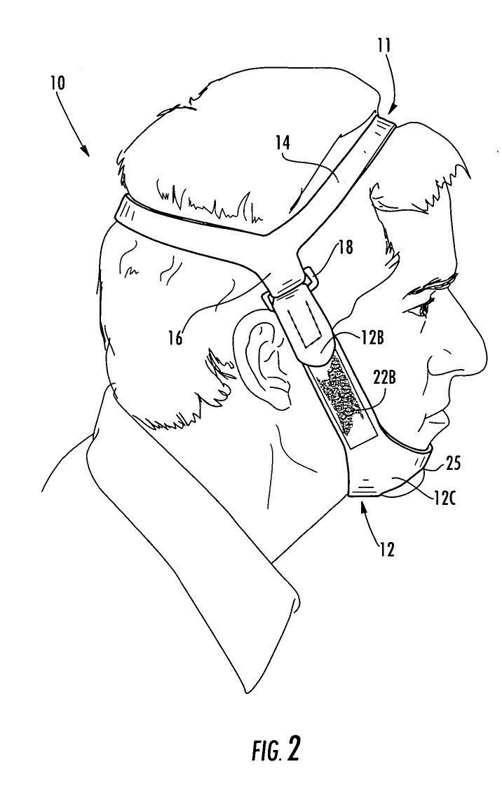

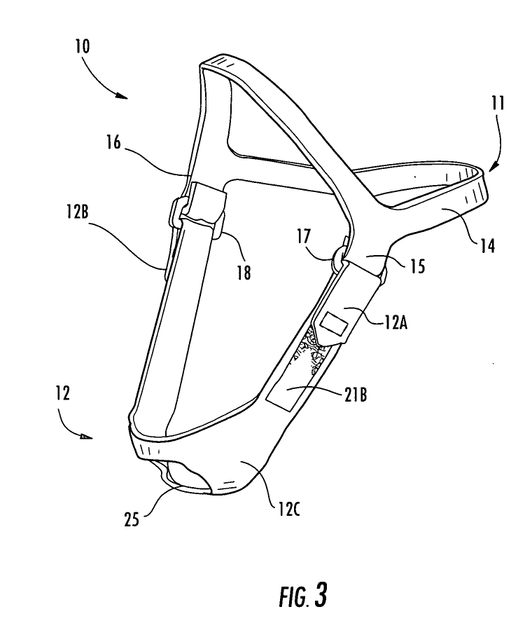

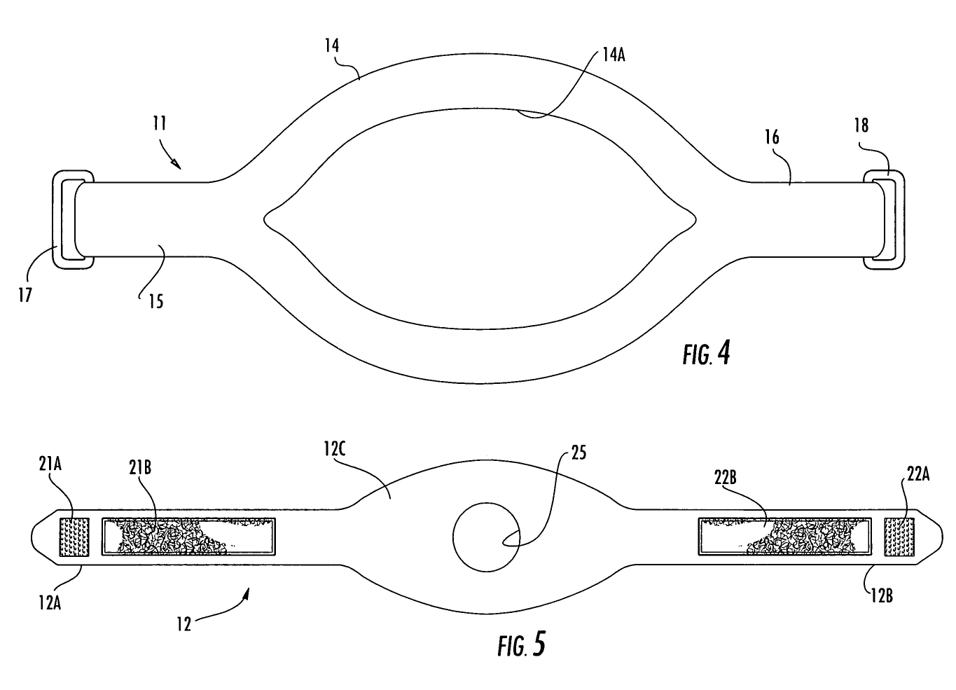

As shown in FIGS. 1 and 2, the present headgear assembly 10 includes a flexible crown 11 and elastic chin strap 12. The crown 11 comprises an elastic closed-end strap 14 which substantially encircles the crown of the head during wear, and defines a crown opening 14A (FIG. 4). Referring to FIGS. 1-4, the crown strap 14 has first and second fastener tabs 15 and 16 extending adjacent the patient's ears and having attached plastic rings 17, 18 designed for receiving respective free ends 12A, 12B of the chin strap 12. Each end 12A, 12B of the chin strap 12 has complementary hook and loop fasteners 21A, 22A and 21B, 22B, as best shown in FIGS. 1 and 5. The hook and loop fasteners 21A, 22A, 21B, 22B reside on an outside surface of the chin strap 12 to avoid irritating the skin of the patient during use. To attach the chin strap 12, the free ends 12A, 12B are fed through respective fastener rings 17, 18, and are then folded over (as demonstrated by arrow 24 in FIG. 1) such that the hook fasteners 21A, 21B releasably mate with the adjacent loop fasteners 22A, 22B. Once attached and applied to the head of the patient, the chin strap 12 comfortably engages the chin to support the lower jaw, and maintain the mouth in a substantially closed position when sleeping.

Preferably, an increased-width, elastic intermediate portion 12C of the chin strap 12 resides adjacent the patient's chin and defines an opening 25 which comfortably receives and accommodates the chin. The chin strap 12 is readily adjusted to either increase or decrease pressure against the chin by pulling more or less of the respective free ends 12A, 12B through the fastener rings 17, 18 and attaching the mating hook and loop fasteners 21A, 21B and 22A, 22B, as previously described. Because the ends 12A, 12B of the chin strap 12 are detachable from crown strap 14, the chin opening 25 can remain properly oriented on the patient through substantially equal length adjustment of both ends 12A, 12B. As the chin strap 12 is tightened, the crown strap 14 effectively distributes pressure against the head and helps prevent inadvertent shifting of the chin strap 12 during sleep.

According to one preferred embodiment, both the crown 11 and chin strap 12 are constructed of a breathable, elastic foam material laminated on both sides with a soft moisture-wicking fabric. Preferably, the outside fabric incorporates Lycra® spandex and an anti-microbial element, such as silver. The width of the crown strap 14 and free ends 12A, 12B of the chin strap 12 is at least one inch.

A headgear assembly is described above. Various details of the invention may be changed without departing from its scope. Furthermore, the foregoing description of the preferred embodiment of the invention and best mode for practicing the invention are provided for the purpose of illustration only and not for the purpose of limitation—the invention being defined by the claims.

I claim:

1. A headgear assembly adapted for wear by a sleep apnea patient and designed for holding a lower jaw of the patient substantially closed during sleep, said headgear assembly comprising:
a flexible crown for residing atop a head of the patient, and comprising a crown-encircling strap and a crown opening defined by said strap; and
a flexible chin strap having first and second ends attached to said crown, and adapted for engaging a chin of the patient and supporting the lower jaw.

2. A headgear assembly according to claim 1, and comprising means for adjusting said chin strap relative to said crown to increase or decrease pressure against the chin of the patient.

3. A headgear assembly according to claim 1, wherein said crown comprises a fastener ring adapted for adjustably receiving the first end of said chin strap.

4. A headgear assembly according to claim 2, wherein the first end of said chin strap comprises complementary hook and loop fasteners, such that said chin strap is adjustably attached to said crown by inserting the first end through said fastener ring, and then folding the first end over to releasably mate the hook and loop fasteners.

5. A headgear assembly according to claim 4, wherein said crown comprises a second fastener ring receiving the second end of said chin strap.

6. A headgear assembly according to claim 5, wherein the second end of said chin strap comprises complementary hook and loop fasteners, such that said chin strap is adjustably attached to said crown by inserting the second end through said second fastener ring, and then folding the second end over to releasably mate the hook and loop fasteners.

7. A headgear assembly according to claim 1, wherein said chin strap comprises a launderable foam material.

8. A headgear assembly according to claim 7, wherein said chin strap further comprises elastic yarns.

9. A headgear assembly according to claim 1, wherein said chin strap defines a chin opening adapted for receiving the chin of the patient.

10. A headgear assembly according to claim 1, wherein said crown comprises a launderable foam material.

11. A headgear assembly according to claim 10, wherein said crown further comprises elastic yarns.

12. A headgear assembly adapted for wear by a sleep apnea patient, and designed for holding a lower jaw of the patient substantially closed during sleep, said headgear assembly comprising:
a flexible crown for residing atop a head of the patient, and comprising a crown-encircling strap and a crown opening defined by said strap;
first and second fastener rings attached to said crown-encircling strap and adapted for residing adjacent respective ears of the patient; and
a flexible chin strap removably attached to said crown and adapted for engaging a chin of the patient and supporting the lower jaw, said chin strap having first and second free ends adjustably received through respective first and second fastener rings.

13. A headgear assembly according to claim 12, wherein said chin strap comprises a launderable fabric material.

14. A headgear assembly according to claim 13, wherein the fabric material comprises elastic yarns.

15. A headgear assembly according to claim 12, wherein said chin strap defines a chin opening adapted for receiving the chin of the patient.

16. A headgear assembly according to claim 12, wherein said crown comprises a launderable fabric material.

17. A headgear assembly according to claim 16, wherein the fabric material comprises elastic yarns.

18. A headgear assembly according to claim 12, wherein the first and second free ends of said chin strap comprise respective hook and loop fasteners.

19. A method for holding a lower jaw of a sleep apnea patient substantially closed during sleep, said method comprising the steps of:
arranging a flexible crown atop a head of the patient, the crown comprising a crown-encircling strap and a crown opening defined by the strap; and
locating a flexible chin strap adjacent a chin of the patient to support the lower jaw, the chin strap being attached to the crown.

20. A method according to claim 13, and comprising adjusting the chin strap relative to the crown to increase or decrease pressure against the chin of the patient.
